(19) United States
(12) Patent Application Publication
(10) Pub. No.: US 2002/0022599 A1
(43) Pub. Date: Feb. 21, 2002

(54) Methods of modulating drug clearance mechanisms by altering SXR activity

(76) Inventors: Timothy W. Synold, Monrovia, CA (US); Isabelle Dussault, Thousand Oaks, CA (US); Barry M. Forman, Newport Beach, CA (US)

Correspondence Address: ROTHWELL, FIGG, ERNST & MANBECK, P.C., 555 13TH STREET, N.W., SUITE 701, EAST TOWER, WASHINGTON, DC, 20004, US

(21) Appl. No.: 09/815,300

(22) Filed: Mar. 23, 2001

Related U.S. Application Data
(60) Provisional application No. 60/191,767, filed on Mar. 24, 2000.
(60) Provisional application No. 60/266,866, filed on Feb. 7, 2001.

(51) Int. Cl. A61K 48/00; A61K 31/337

(52) U.S. Cl. 514/44; 514/449

(57) ABSTRACT

The present invention relates to new methods of modifying drug clearance and avoiding multi-drug resistance by modifying SXR activity. SXR is a transcriptional activator of MDR1, cytochrome P40-3A4 and cytochrome P40 2C8. SXR activation can significantly increase the metabolic inactivation and efflux of a wide range of chemotherapeutic agents, for example taxanes. Reducing and/or preventing SXR activation therefore diminishes drug resistance and drug clearance and forms the basis of important therapeutic methods which increase the performance of drugs, such as taxanes. Screening and drug identification methods are described which can identify drugs which are not susceptible to SXR related inactivation and increased efflux. In addition, drugs which can reduce these effects for other agents are provided.

### CROSS-REFERENCE TO RELATED APPLICATIONS

This application claims priority from provisional application serial no. 60/191,767, filed Mar. 24, 2000 and provisional application serial no. 60/266,866, filed Feb. 7, 2001.

### GOVERNMENT RIGHTS

This invention was made in part under grant no. CA 33572 from the United States National Cancer Institute. The United States government has certain rights in the invention.

### BACKGROUND OF THE INVENTION

1. Technical Field

This invention generally pertains to the field of modulating nuclear hormone receptor SXR and screening for SXR activity, expression and effects to provide novel methods and compounds related to influence on and detection of drug clearance mechanisms.

2. Description of the Background Art

The effectiveness of many pharmacologic agents are limited by metabolic inactivation and excretion. The metabolism of paclitaxel (Taxol), one of the most commonly used antineoplastic agents, exemplifies the effect of these natural clearance pathways on drug efficacy. Paclitaxel and many other drugs, including, but not limited to HIV protease inhibitors, Tamoxifen, trans retinoic acid, Tolbutamide, Atovastatin, Gemfibrozol, Amiodarone, Anastrozole, Azithromycin, Cannabinoids, Cimetidine, Clarithromycin, Clotrimazole, Cyclosporine, Danazol, Delavirdine, Dexamethasone, Diethyldithiocarbamate, Diltiazem, Dirithromycin, Disulfiram, Entacapone, Erythromycin, Ethinyl estradiol, Fluconazole, Fluoxetine, Fluvoxamine, Gestodene, Grapefruit juice, Indinavir, Isoniazid, Itraconazole, Ketoconazole, Metronidazole, Mibefradil, Miconazole, Nefazodone, Nelfinavir, Nevirapine, Norfloxacin, Norfluoxetine, Omeprazole, Oxiconazole, Paroxetine, Propoxyphene, Quinidine, Quinine, Quinupristin, Dalfopristin, Ranitidine, Ritonavir, Saquinavir, Sertindole, Sertraline, Troglitazone, Troleandomycin, Valproic acid, Verapamil, Zafirlukast and Zileuton, are subject to metabolic inactivation by the hepatic cytochrome P450 enzymes CYP3A4 and CYP2C8. Both enzymes hydroxylate paclitaxel, thereby abolishing the drug's antimitotic properties. See Monsarrat et al., Bull. Cancer 84:125-133, 1997; Kearns, Pharmacother. 17:105S-109S, 1997; Crommentuyn et al., Cancer Treat. Rev. 24:345-366, 1998. In addition to being inactivated by hepatic P450 enzymes, drugs also are excreted from the intestine by P-glycoprotein (ABCB1), a broad specificity efflux pump that is the product of the MDR1 gene. Gene targeting studies have demonstrated that P-glycoprotein is responsible for the fecal excretion of 85% of orally administered paclitaxel. Sparreboom et al., Proc. Natl. Acad. Sci. USA 94:2031-2035, 1997. Moreover, when overexpressed in tumor cells, P-glycoprotein establishes a barrier to the uptake of paclitaxel and other agents by the tumor, creating the therapeutic obstacle of multidrug resistance. Ambudkar et al., Annu. Rev. Pharmacol. Toxicol. 39:361-398, 1999.

CYP3A4 is a critical enzyme in the oxidative metabolism of a wide variety of xenobiotics. Due to its abundance in the liver and intestine and its broad substrate specificity, CYP3A4 is involved in the biotransformation of more than 60% of clinically used drugs including anti-epileptics, immunosuppressives, antimycotics, and antibiotics. Maurel, in Ionnides, ed. Cytochromes P450: Metabolic and Toxicological Aspects. Boca Raton, Fla.: CRC Press, Inc., pp. 241-270, 1996. CYP3A4 is also involved in the catabolism of several anticancer agents including taxanes, epipodophyllotoxins, and vinca alkaloids. Harris et al., Canc. Res. 54:4026-4035, 1994; Royer et al., Canc. Res. 56:58-65, 1996; Zhou-Pan et al., Canc. Res. 53:5121-5126, 1993; Krikorian et al., Semin. Oncol. 16:21-25, 1989. Furthermore, CYP3A4 plays a major role in the metabolism of the clinically useful antiestrogens tamoxifien and toremifene. Mani et al., Carcinogen. 15:2715-2720, 1994; Berthou et al., Biochem. Pharmacol. 47:1883-1895, 1994. CYP3A4 is known to be highly inducible both in vitro and in vivo, resulting in many clinically significant drug-drug interactions. Williams et al., Biochem. Soc. Trans. 22:131S, 1994; Kovacs et al., Clin. Pharmacol. Ther. 63:617-622, 1998. Its transcription can be induced by many of its substrates. Saras et al., Mol. Pharmacol. 56:851-857, 1999. The orphan nuclear receptor, (“SXR”) (also known as PXR, PAR, PRR, NR1I2), plays a central role in regulating CYP3A4 transcription. Saras et al., Mol. Pharmacol. 56:851-857, 1999; Kliewer et al., Cell 92:73-82, 1998; Blumberg et al., Genes Dev. 12:3195-3205, 1998; Bertilsson et al., Proc. Natl. Acad. Sci. USA 95:12208-12213, 1998; Lehmann et al., J. Clin. Invest. 102:1016-1023, 1998.

SXR is a nuclear receptor shown to respond to a wide variety of natural and synthetic compounds, as well as to some commonly used pharmacologic agents including, for example, rifampicin, SR12813, clotrimazole, hyperforin and RU486. Jones et al., Mol. Endocrinol. 14:27-39, 2000; Moore et al., Proc. Natl. Acad. Sci. USA 97:7500-7502, 2000; Wentworth et al., J. Endocrinol. 166:R11-R16, 2000. Recent gene targeting and transgene studies have confirmed that activation of SXR promotes CYP3A4 expression in the liver. Xie et al., Nature 406:435-439, 2000. Thus SXR is a highly promiscuous xenobiotic sensor that plays a critical role in regulating hepatic drug metabolism. SXR is also highly expressed in the intestine; its role in this organ is not fully understood.

Nuclear receptors such as SXR are ligand-modulated transcription factors that mediate the transcriptional effects of steroid and related hormones. These receptors have conserved DNA-binding domains (DBD) which specifically bind to the DNA at cis-acting elements in the promoters of their target genes and ligand binding domains (LBD) which allow for specific activation of the receptor by a particular hormone or other factor. Transcriptional activation of the target gene for a nuclear receptor occurs when the ligand binds to the LBD and induces a conformation change in the receptor that facilitates recruitment of a coactivator or displacement of a corepressor. This results in a receptor complex which can modulate the transcription of the specific gene. Recruitment of a coactivator after agonist binding allows the receptor to activate transcription. Binding of a receptor antagonist induces a different conformational change in the receptor such that there is no interaction or there is a non-productive interaction with the basal transcriptional machinery of the target gene. As will be apparent to those skilled in the art, an agonist of a receptor that effects negative transcriptional control over a particular gene will actually decrease expression of the gene. Conversely, an antagonist of such a receptor will increase expression of a negatively regulated gene.

Northern blot analysis of SXR revealed that it is abundantly expressed in the liver and small and large intestine. Blumberg et al., Genes Dev. 12:3195-3205, 1998; Bertilsson et al., Proc. Natl. Acad. Sci. USA 95: 12208-12213, 1998; Lehmann et al., J. Clin. Invest. 102:1016-1023, 1998. Recent reports suggest SXR is variably expressed in human tumors such as neoplastic breast tissue. See Dotzlaw et al., Clin. Canc. Res. 5:2103-2107, 1999. Although no obvious differences in levels of SXR expression between normal and neoplastic breast tissue were detected, the RT-PCR method used was not considered quantitative. The authors also reported that in a panel of human breast cancer cell lines, four out of six expressed SXR with an apparent wide range of mRNA levels.

In response to known activators, SXR induces transcription of the major hepatic and intestinal monooxygenase enzyme, cytochrome P450 3A4 (CYP3A4). CYP3A4 is the most abundant cytochrome P450, comprising about 25% of all cytochromes P450, and is responsible for the primary metabolic inactivation of many drugs. Like SXR, CYP3A4 is expressed in liver and intestine and can also be found in some human tumors (Murray et al. Br. J. Cancer 1999). SXR, therefore, represents a sensor in a new signaling pathway that controls activation of drug metabolism both in normal and malignant tissues.

SXR can activate reporter constructs which contain response elements from several cytochrome P450 (CYP) genes that encode enzymes involved in the metabolism of both natural and synthetic compounds. In response to known activators, SXR binds to a specific nuclear receptor response element in the promoter of CYP3A4 as a heterodimer with the retinoid X receptor (RXR), leading to transcriptional activation. See FIG. 1A. The SXR/RXR complex is activated by rifampicin, hyperforin, and wide variety of structurally diverse compounds previously shown to modulate expression of CYP3A4. Lehmann et al., J. Clin. Invest. 102:1016-1023, 1998.

The CYP3A4 promoter has been cloned and some of its transcriptional regulatory elements have been identified. For example, an approximately 20-bp region approximately 150-bp upstream of the transcription start site confers responsiveness to SXR agonists. Barwick et al., Mol. Pharmacol. 50:10-16, 1996; Hashimoto et al., Eur. J. Biochem. 218:585-595, 1993. This region contains two copies of a degenerate motif known to be recognized by members of the nuclear receptor superfamily. Several groups have recently identified SXR as the orphan nuclear receptor that interacts with the response element in the CYP3A4 promoter leading to transcriptional activation. Blumberg et al., Genes Dev. 12:3195-3205, 1998; Bertilsson et al., Proc. Natl. Acad. Sci. USA 95:12208-12213, 1998; Lehmann et al., J. Clin. Invest. 102:1016-1023, 1998.

MDR1, like CYP3A4, is a critical gene in the detoxification pathway of xenobiotics. MDR1 encodes P glycoprotein (Pgp), a multidrug transporter that removes a variety of drugs and chemotherapeutic agents from the plasma membrane to the outside of a cell. Consistent with their role in detoxification, both CYP3A4 and Pgp are most highly expressed in the tissues that participate in drug metabolism and elimination, such as liver and intestine. Thiebaut et al., Proc. Natl. Acad. Sci. USA 84:7735-7738, 1987; Watkins et al., J. Clin. Invest. 80:1029-1036, 1987. Moreover, many substrates or modulators of CYP3A4 are also substrates or modulators of Pgp. Wacher et al., Mol. Carcinogen. 13:129-134, 1995. Efficient inducers of CYP3A4, such as rifampicin, phenobarbital, and clotrimazole also activate the transcription of MDR1. Schuetz et al., Mol. Pharmacol. 49:311-318, 1996. This significant overlap in substrate/inducer specificity suggests that CYP3A4 and MDR1 are co-regulated, and therefore act in concert to detoxify and deactivate a wide range of compounds.

The two commercially available members of taxane class of anticancer drugs, paclitaxel and docetaxel, are among the most active agents in the treatment of breast, ovarian, and non-small cell lung cancer. Paclitaxel is metabolized in the liver by two routes, CYP3A4 and cytochrome P450 2C8 (CYP2C8). Both CYP2C8 and CYP3A4 may contribute to paclitaxel inactivation in man (Kostrubsky et al., Arch. Biochem. Biophys., 1998). Docetaxel is almost exclusively metabolized by CYP3A4 (Royer et al., Cancer Res. 1996).

In humans, taxol is converted to inactive metabolites through interactions with CYP2C8 and CYP3A4. Harris et al., Canc. Res. 54:4026-4035, 1994; Rahman et al., Canc. Res. 54:5543-5546, 1994. Although some investigators have concluded that oxidative metabolism via CYP2C8 is the principal route of taxol inactivation, most studies have been performed using microsomal preparations or intact hepatocytes from donors with unknown past medical histories. In the study by Sonnichsen et al., CYP2C8 was not the predominant route of taxol metabolism in some of the primary hepatocyte cultures studied. Sonnichsen et al., J. Pharmacol. Exp. Ther. 275:566-575, 1995. A subset analysis of hepatocytes obtained from patients with detailed donor histories revealed that 13-hydroxytaxol formed via CYP3A4, was the predominant metabolite in donors who had received phenobarbital. Therefore, CYP3A4 is an important enzyme in the biotransformation of taxol, particularly in patients receiving concomitant CYP3A4 inducers or very high doses of taxol. Recent reports have shown that CYP2C8 is implicated in the degradation of a variety of clinically significant drugs including paclitaxel, all trans retinoid acid, tolbutamide, azidothymidine, verapamil, ibuprofen, thiazolidinediones, benzodiazepines and others (Smith et al., Xenobiotica 28:1095-1128, 1998); Goldstein and de Morais, Pharmacogenetics 4:285-299, 1994).

In primary human hepatocytes, taxol induces immunoreactive CYP3A4 protein and mRNA levels at pharmacologically relevant concentrations. Kostrubsky et al., Arch. Biochem. Biophys. 355:131-136, 1998. Furthermore, taxol increases CYP3A4 enzyme activity. This effect is concentration-dependent, with maximal increase in enzyme activity observed at 10 μM taxol.

While xenobiotic compounds are routinely cleared by metabolic inactivation, other mechanisms exist to purge the body of potentially toxic foreign compounds. In fact, inhibition of xenobiotic uptake would be a more logical first line of defense. P-glycoprotein, the product of the MDR1 gene (ABCB1) is a broad-specificity xenobiotic transporter that inhibits uptake and subsequent exposure to a wide variety of foreign compounds. See Ambudkar et al., Annu. Rev. Pharmacol. Toxicol. 39:361-398, 1999.

MDR1 and its gene product Pgp are over-expressed in a wide range of human tumors both de novo and following treatment with Pgp substrates in vivo. Goldstein et al., J. Natl. Canc. Inst. 81:116-124, 1989; Fojo et al. Proc. Natl. Acad. Sci. USA 84:265-269, 1987; Beck et al., Canc. Res. 56:3010-3020, 1996; Chan et al., N.E.J.M. 325:1608-1614, 1991; Picker et al., J. Natl. Canc. Inst. 83:708-712, 991; Marie et al., Blood 78:586-592, 1991. The widely held belief in the importance of MDR1 as a determinant of clinical drug sensitivity has been underscored by the enormous resources that have been dedicated to finding ways to reverse Pgp function in patients. Beck et al., Canc. Res. 56:3010-3020, 1996.

Much of the previous work investigating the importance of MDR1 in drug resistance has concentrated on whether stable over-expression of MDR1 results in clinical resistance. More recently, others have proposed that a static determination of MDR1 expression ignores transient expression changes that may be an important determinant of Pgp-mediated resistance. Abolhoda et al. have shown that MDR1 expression is rapidly activated in human tumors in vivo following exposure to chemotherapy. Abolhoda et al., Clin. Canc. Res. 5:3352-3356, 1999. These authors conclude that transcriptional regulation, rather than gene amplification, may be a more important determinant of MDR1-mediated drug resistance in vivo.

### SUMMARY OF THE INVENTION

This invention provides a method of modifying drug pharmacokinetics which comprises altering the activity of SXR on expression levels of CYP2C8 or MDR1. The invention also provides a method of modifying multiple drug resistance which comprises altering SXR activity. Embodiments of these methods include those wherein drug catabolism is altered (reduced or increased), wherein drug intestinal efflux is altered (reduced or increased), wherein drug oral absorption is altered (reduced or increased) and wherein biliary excretion is altered (reduced or increased). The invention provides embodiments of the methods which comprise altering SXR mRNA levels, SXR protein levels, the ability of SXR to recruit coactivator or the displacement of corepressor from SXR. Additional embodiments are provided in which the drug is a taxane. Further, the invention provides methods which comprise administering an SXR antagonist, such as ecteinascidin-743 or an 8XR agonist. In addition, methods are provided which comprise administering a ribozyme which cleaves mRNA encoding SXR, an SXR coactivator or a SXR corepresser. Further methods include those which comprise administering an antisense oligonucleotide which suppresses transcription or translation of SXR, an SXR coactivator or an SXR corepressor.

The invention further provides a method of identifying drugs with improved pharmacokinetic properties or activity which comprises screening drug candidates for their ability to modulate SXR. Embodiments of this method include those which comprise identifying drugs having altered efflux characteristics by screening drug candidates for their ability to modulate the activity of SXR on expression levels of CYP2C8 or MDR1. Methods also include those which comprise identifying drugs having altered catabolism by screening drug candidates for their ability to modulate the activity of SXR on expression levels of CYP2C8 or MDR1. Further embodiments include those which comprise identifying drugs having altered oral bioavailability or biliary excretion by screening drug candidates for the ability to modulate the activity of SXR on expression levels of CYP2C8 or MDR1.

The invention also provides embodiments wherein the drug candidates screened in the methods described above are taxanes. The invention provides methods which comprise monitoring SXR activity in cells in vivo or in vitro according to the methods described above.

Methods such as those described above include those wherein the monitoring of SXR activity comprises monitoring the expression of an endogenous SXR regulated gene such as CYP3A4, CYP2C8 and MDR1. In addition, the invention provides methods such as those described above wherein the monitoring of SXR activity comprises monitoring the expression of a synthetic reporter gene under the control of control elements responsive to SXR or the expression of a chimeric gene wherein the protein encoded by the chimeric gene maintains the ability to respond to SXR ligands.

The invention also provides specific embodiments wherein the monitoring of SXR activity comprises monitoring coactivator recruitment, corepressor displacement, SXR/RXR interaction, and SXR binding or SXR/RXR binding to DNA response elements in regulatory sequences that control expression of CYP2C8, CYP3A4 or MDR1 genes or to nucleotide sequences that bind to SXR or the SXR/RXR complex.

The invention also provides a method of identifying drugs that do not modulate SXR activity which comprises screening drug candidates for their inability to modulate the activity of SXR on expression levels of CYP2C8 or MDR1, modulate the expression of CYP3A4, modulate the expression of CYP2C8, modulate the expression of MDR1, modulate the expression of a synthetic reporter gene under the control of control elements responsive to SXR, modulate the expression of a chimeric gene wherein the protein encoded by the chimeric gene maintains the ability to respond to SXR ligands, modulate SXR coactivator recruitment; modulate SXR corepressor displacement, modulate SXR or SXR/RXR complex binding to DNA response elements in regulatory sequences that control expression of CYP2C8, CYP3A4 or MDR1 genes or modulate SXR/RXR interaction.

The invention also provides drugs identified by any of the methods described above.

The invention provides a method of screening patients to predict responsiveness to a pharmacologic agent, which comprises obtaining a biological sample from the patient and screening said biological sample for an SXR parameter selected from the group consisting of SXR mRNA levels, SXR protein levels, SXR coactivator levels, SXR-coactivator interactions, SXR corepressor levels, SXR-corepressor interactions, SXR polymorphisms, SXR mutations, expression of an endogenous SXR regulated gene and levels of an endogenous SXR ligand. Preferred embodiments of this method include those in which the biological sample is screened for expression of an endogenous SXR regulated gene such as CYP3A4 and CYP2C8. The responsiveness to a pharmacologic agent is responsiveness to a therapeutic effect, a toxic effect or a drug interaction. Pharmacologically agents may be selected from an endogenous compound or from exogenous compounds such as a drug, an herbal compound and a nutrient. The biological sample tested in such methods may be a tumor sample or normal cells or tissues, or materials derived from them.

The invention provides a method of drug chemotherapy which comprises coadministering a drug and an agent that modulates (upregulates or downregulates the activity or expression of SXR. The invention further provides a method of increasing the effectiveness of a drug which comprises coadministering the drug with an agent that modulates the actions of SXR. Embodiments of the above methods include those wherein the agent is an SXR antagonist, an SXR agonist or wherein the agent does not activate SXR. Further embodiments include those wherein the drug is a taxane.

### BRIEF DESCRIPTION OF THE DRAWINGS

FIG. 1A provides a schematic diagram showing the binding of the SXR receptor onto a CYP3A4 response element.

FIG. 1B illustrates mechanisms involved in drug clearance.

FIG. 2 shows the activation of Gal-L-SXR and Gal-L-RXR after activation by SXR agonists.

FIG. 3 is a bar graph showing the activation of the indicated nuclear hormone receptor by 10 micromolar paclitaxel.

FIG. 4 is a northern blot showing the expression of the indicated genes in primary human hepatocytes and human LS180 intestinal cells in response to rifampicin, SR121813, paclitaxel and LG268.

FIG. 5 is a bar graph showing the activation of a reporter construct containing SXR response elements from the CYP3A4 promoter by a constitutively active variant of SXR (VP-SXR).

FIG. 6 is a northern blot showing the induction of expression of the indicated genes by VP-SXR.

FIG. 7 provides data showing the fold activation of the Gal-L-SXR report gene in CV-1 cells treated with paclitaxel and docetaxel.

FIG. 8 is a northern blot showing the expression of the indicated genes in primary human hepatocytes and human LS180 cells in response to treatment with paclitaxel and docetaxel.

FIG. 9 is a western blot using a P-glycoprotein antibody of human LS180 cells treated with paclitaxel or docetaxel.

FIG. 10 is a bar graph showing results of the 3′-p-hydroxypaclitaxel production after induction by the indicated drugs.

FIG. 11 presents data on paclitaxel efflux in human LS180 cells after induction by the indicated drugs.

FIG. 12 shows the results of a mammalian two hybrid assay comparing the effects of the paclitaxel and docetaxel on co-regulator recruitment.

FIG. 13 shows the inhibitory activity of SXR in the absence of ligand.

FIG. 14 presents data regarding the interaction of SXR with corepressors in the presence of paclitaxel or docetaxel.

FIG. 15 presents data showing that ecteinascidin-743 antagonizes SXR activity.

FIG. 16 is a bar graph showing reporter activity data in CV-1 cells transfected with an LXREₓ3-TK-Luc reporter and an expression vector for CARβ and treated with androstanol (Anol) or ET-743 (ET).

FIG. 17 is a graph showing dose response studies for inhibition of SXR by ET-743.

FIG. 18 is a northern blot showing that ET-743 inhibited drug induced activation of CYP3A4 and MDR1.

FIG. 19 is a representative polyacrylamide gel showing the expression of SXR, MDR1 and CYP3A4 in a panel of human tumor cell lines.

### BRIEF DESCRIPTION OF THE PREFERRED EMBODIMENTS

Using a combination of pharmacologic and genetic approaches, we demonstrate that SXR activates MDR1 expression in primary human hepatocytes and intestinal cells and show that this activation results in enhanced drug efflux. These findings provide the first evidence that SXR coordinately regulates multiple xenobiotic clearance pathways (metabolism and excretion) in different tissues (intestine and liver) . It is interesting to note that SXR and P-glycoprotein are co-expressed in a number of tissues including hepatocytes, intestinal epithelia, kidney, and the placenta. See Sparreboom et al., Proc. Natl. Acad. Sci. USA 94:2031-2035, 1997; Ambudkar et al., Annu. Rev. Pharmacol. Toxicol. 39:361-398, 1999; Jones et al., Mol. Endocrinol. 14:27-39, 2000. P-glycoprotein expression has also been detected in the capillary endothelial cells that form the blood-brain and blood-testis barriers. Together, this suggests that SXR may contribute to drug excretion by the kidney, and to protecting the brain and fetus from exposure to toxic compounds. See Ambudkar et al., Mol. Endocrinol. 39:361-398, 1999.

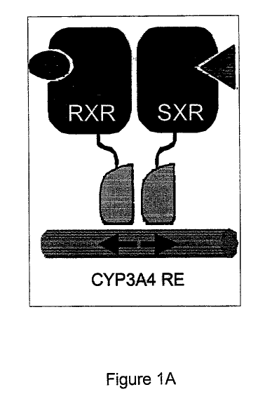

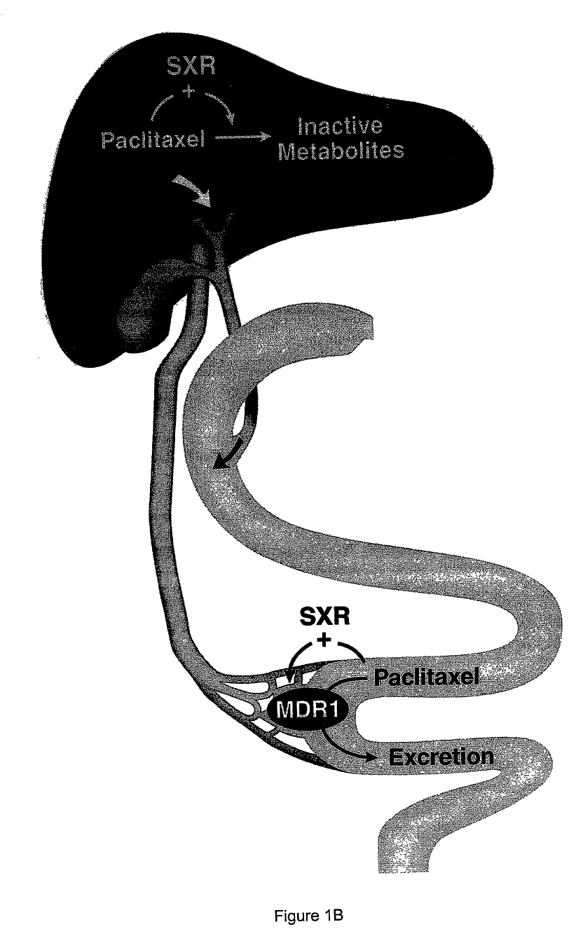

SXR Coordinately Regulates Drug Metabolism and Efflux. The response to a xenobiotic challenge is illustrated with paclitaxel, a naturally occurring chemotherapeutic agent that can be cytotoxic to a wide variety of cells. Oral exposure to paclitaxel results in activation of SXR in intestinal epithelial cells. This results in enhanced expression of the MDR1/P-glycoprotein transporter and subsequent excretion of paclitaxel into intestinal fluid. In principle, any paclitaxel that may pass this barrier could be transported to the liver via the portal vessels and eventually enter the general circulation. However, paclitaxel is hydroxylated by CYP3A4, a modification that destroys the cytotoxic properties of this drug. CYP3A4 is expressed in the intestine and liver and is induced by SXR. In addition, CYP2C8, another paclitaxel-inactivating enzyme, is also induced by SXR in the liver. The inactivated paclitaxel metabolites can then be secreted into the biliary fluid and then removed from the gastrointestinal tract. Thus, in response to a xenobiotic challenge, SXR can induce both a first line of defense (intestinal excretion) and a back-up system (hepatic inactivation) that limits exposure to potentially toxic compounds. While this system can limit exposure to environmental toxins, it can create a therapeutic problem when it limits the bioavailability of pharmaceutical compounds and in particular the oral bioavailability of these compounds. Similarly, this regulatory loop could prevent cell-killing by chemotherapeutic agents should it be activated in a tumor. See FIG. 1B.

Despite the similarities between paclitaxel and docetaxel, resistance to the two drugs does not always occur through a common pathway. Paclitaxel, but not docetaxel, can activate SXR and induce the transcription of a reporter gene containing response elements from the CYP3A4 gene and induces CYP3A4 expression and activity through SXR. Transcription of the endogenous CYP3A4 gene is strongly induced in primary human hepatocytes treated with paclitaxel, but not docetaxel. Furthermore, only paclitaxel strongly induces CYP3A4 activity and subsequently its own metabolism.

These findings have important implications in the treatment of taxane-responsive tumors and suggest that differences in SXR responsiveness can predict clinical outcome. Tumor cells, or normal cells or tissues, can be removed from a cancer patient who is a candidate for taxane therapy, and the cells tested for presence of SXR above a threshold level, for SXR polymorphisms or for SXR mutations. For example, the cells can be tested for presence of SXR protein by antibody binding, using a polyclonal or monoclonal anti-SXR antibody. Alternatively, the cells can be tested for presence of SXR mRNA, for example, by reverse transcription polymerase chain reaction. Presence of SXR above the threshold level indicates that the patient will likely respond better to treatment with an SXR non-activator such as docetaxel than to treatment with an SXR activator such as paclitaxel. Other mRNA detection methods include any suitable method known in the art.

The demonstration that paclitaxel activates SXR, which subsequently leads to a coordinate increase in the expression of genes required for drug clearance, implies that anti-cancer chemotherapeutic agents or any pharmacological agents which activate SXR, enhance clearance of drugs that are substrates for CYP3A4, CYP2C8 and/or P-glycoprotein. Therefore, taxanes and other chemotherapeutic agents may exhibit enhanced efficacy or become bioavailable after an oral dose if they do not activate SXR. A method to screen taxanes and other known or potential chemotherapeutic agents for the ability to activate SXR can identify chemotherapeutic agents which do not activate SXR and thus have preferred pharmacokinetic properties, especially in persons susceptible to multidrug resistance.

Paclitaxel is an SXR activator that induces hepatic expression of CYP2C8 as well as CYP3A4. Thus the genetic targets of SXR activation include cytochrome P450 2C8. SXR also activates MDR1 expression in intestinal tumor cells, causing enhanced paclitaxel efflux. Importantly, these results show that SXR responses include both intestinal drug excretion and multidrug resistance. The ability of paclitaxel to activate SXR implies that the effectiveness of this drug could be limited by autoinduced metabolism, MDR1-mediated clearance and/or multidrug resistance. This implies that the therapeutic activity of taxanes or any SXR activating drugs can be improved in analogs that lack SXR agonist activity. The ability of SXR to coordinately regulate multiple xenobiotic clearance pathways in different tissues shows that this receptor can be exploited to select drug candidates that either fail to activate, or even inhibit these clearance pathways. This invention allows the identification drugs that exhibit both types of activities, and manipulation of SXR responses in a clinical setting. This method, for example, can be used to discover or synthesize drugs which are bioavailable after an oral dose when previous known analogs were not, due to the activation of Pgp via SXR.

Paclitaxel activates SXR at concentrations that are clinically relevant (EC₅₀≈5 μM) and which match the Km for degradation of paclitaxel by CYP3A4 and CYP2C8 (Km≈10 μM). Activation of SXR by paclitaxel results in enhanced expression of CYP3A4, CYP2C8 and P-glycoprotein. This regulatory loop is significant since P-glycoprotein is highly effective in preventing paclitaxel uptake from the intestine. See FIG. 1B. Any paclitaxel that does not enter the bloodstream is ultimately subject to hepatic metabolism (CYP3A4, CYP2C8) and biliary excretion (P-glycoprotein), both of which are induced by SXR. See FIG. 1B.

Overexpression of MDR1 is highly problematic in cancer chemotherapy because it leads to the development of drug resistant tumors. The ability of SXR to induce MDR1 implies that SXR can promote resistance to any chemotherapeutic agent that is a substrate for P-glycoprotein. For example, paclitaxel induces its own efflux from LS180 colon cancer cells. Thus, in addition to regulating traditional drug clearance pathways, SXR may also regulate multidrug resistance in SXR-expressing tumors. Classifying tumors as “SXR-positive” or “SXR-negative” are warranted since this information can predict the likelihood that any particular tumor will develop chemotherapy-induced drug resistance.

The ability of a drug to induce SXR-mediated clearance can limit the therapeutic potential of both the drug which induces the clearance and also any coadministered compounds. Drug-drug interactions can be particularly problematic in many disease therapies, such as cancer chemotherapy, where combinations of drugs are widely used since the activation of SXR by one or more administered drugs can result in increased clearance of other drugs, nutrients or other compounds. Therefore “SXR-transparent” drugs offer therapeutic advantages to their SXR-inducible counterparts. For example, the taxane analog docetaxel failed to activate SXR. The SXR-transparent properties of this drug could not be accounted for solely by an inability to recruit coactivator. Rather, the drug failed to displace corepressors. Since it is well known that β-tubulin is the molecular target for the antineoplastic activities of both of the taxanes, it appears that the chemical structural differences between paclitaxel and docetaxel define a pharmacophore that can be selectively manipulated to minimize SXR responsiveness, a clinically significant finding since docetaxel also failed to induce SXR-mediated drug metabolism and excretion. Taxol is an activator of SXR; taxol activation of SXR leads to induction of CYP3A4 expression and activity; taxol activation of SXR leads to induction of MDR1 expression and activity; and SXR, MDR1, and CYP3A4 are variably expressed in a range of human tumor cell lines.

These new findings lead to the prediction that docetaxel, an SXR-transparent drug, should have improved pharmacokinetic properties over paclitaxel. Clinical studies bear this out: Docetaxel has longer plasma and intracellular half-lives than paclitaxel. Crown et al., Lancet 355:1176-1178, 2000; Eckardt, Am. J. Health Syst. Pharm. 54:S2-S6, 1997. Ligands for nuclear hormone receptors activate transcription by initiating an exchange among coregulatory proteins that associate with the receptor. In the absence of ligand, some receptors associate with a repressor complex that uses the corepressors SMRT or NCoR to dock to the receptor surface. Ligand binding to the receptor results in a reorientation of the receptor transactivation domain such that it displaces the corepressor and simultaneously recruits a number of coactivator proteins including members of the p160 family (SRC-1, ACTR, GRIP) and PBP (DRIP205, TRAP220). The inability of docetaxel to activate SXR-mediated drug clearance demonstrates the utility of developing drugs that fail to activate SXR (“SXR-transparent” drugs).

In summary, the data provided here show that SXR coordinately regulates a network of xenobiotic clearance genes in both the liver and intestine. This places SXR at a critical node in drug clearance pathways. SXR therefore can be used to identify compounds that differentially modulate these pathways to improve the pharmacokinetic properties of drugs, including bioavailability, oral bioavailability, biliary excretion and drug interactions which affect those properties of coadministered drugs. It is an ideal molecular target for the manipulation of this signaling network.

In summary, paclitaxel can activate SXR, while at the same concentration, the structurally related compound, docetaxel, is a much less effective activator. SXR activation by paclitaxel results in increased expression of CYP3A4, CYP2C8, and MDR1. SXR ligands upregulate CYP2C8 in the liver and MDR1 in both the liver and intestine. The discovery of MDR1 as an SXR target gene extends the biological properties of SXR to include the regulation of drug excretion and metabolism, affecting such clinically important factors as in vivo drug resistance in tumors and the bioavailability of oral dosage forms of many drugs. The development of drugs that do not activate SXR would not only limit their metabolism but would also lower biliary and intestinal excretion allowing better availability of poorly absorbed drugs and even allowing oral absorption of drug classes which previously were not bioavailable after an oral dose. The extension of SXR action to the intestine (up-regulation of CYP3A4 and MDR1) demonstrates that SXR is a “master” regulator of drug clearance (metabolism and excretion) in both the liver and the intestine. Thus, for example, activation of SXR by paclitaxel would lead to an enhanced rate of metabolic inactivation in the liver (via CYP3A4 and CYP2C8), enhanced biliary excretion (via MDR1) and decreased absorption in the intestine.

On the other hand, some drugs require activation by P450 cytochrome enzymes such as CYP2C8. These drugs may advantageously be coadministered with a drug that activates SXR (such as an SXR agonist) to increase their activity. Therefore SXR agonist also may be used to beneficially modulate a drug's pharmacokinetic properties, and this invention contemplates their use.

Recent studies have identified a novel marine-derived low molecular weight, hydrophobic natural product, ecteinascidin-743 (ET-743) as an extremely potent antineoplastic agent which inhibits the proliferation of a variety of cancer cell-lines and human xenografts with IC_{50S} ranging from 1-100 nM. Valoti et al., Clin. Canc. Res. 4:1977-1983, 1998; Rinehart, Med. Res. Rev. 20:1-27, 2000; Hendriks et al., Ann. Oncol. 10:1233-1240, 1999; Izbicka et al., Ann. Oncol. 9:981-987, 1988; Martinez et al., Proc. Natl. Acad. Sci. USA 96:3496-3501, 1999. Although the mechanism of action of this drug is unclear, its high potency implies that it acts through a specific molecular target. ET-743 has been shown to inhibit trichostatin-induced transcription of MDR1. Minuzzo et al., Proc. Natl. Acad. Sci. USA 97:6780-6784, 2000; Jin et al., Proc. Natl. Acad. Sci. USA 97:6775-6779, 2000.

In the case of cancer chemotherapy in particular, MDR1 expression establishes significant barriers to effective treatment. In addition to MDR1 effects on drug efflux, P-glycoprotein may inhibit cells from undergoing apoptosis directly. Ruth et al., Canc. Res. 60:2576-2578, 2000; Pallis et al., Blood 95:2897-2904, 2000. Thus, in addition to developing SXR-transparent drugs, there is significant therapeutic value in identifying SXR antagonists that inhibit MDR1 expression. For example, ET-743 antagonizes SXR at nanomolar concentrations. The identification of a compound that inhibits SXR-mediated drug clearance pathways suggests a molecular approach to develop pharmaceutical reagents that enhance therapeutic efficacy. This permits the use of lower doses of conventional chemotherapeutic agents, a practice which will lower costs and minimize the cytotoxic side effects of these drugs.

All mammalian expression vectors contained the cytomegalovirus promoter/enhancer followed by a bacteriophage T7 promoter for transcription in vitro. The following full-length proteins were expressed in this vector; human SXR (accession AF061056) and mouse CARβ (accession AF009327). Gal4 fusions containing the indicated protein fragments were fused to the C-terminal end of the yeast Gal4 DNA binding domain (amino acids 1-147, accession X85976), Gal-L-SXR (human SXR ligand binding domain, Lys 107-Ser 443, accession AF061056), Gal-L-RXR (human RXRα ligand binding domain, Glu 203-Thr 462, accession X52773), Gal-SRC1 (human SRC-1, Asp 617-Asp 769, accession U59302), Gal-ACTR (human ACTR, Ala 616-Gln 768, accession AF036892), Gal-GRIP (mouse GRIP1, Arg 625-Lys 765, accession U39060), Gal-PBP (human PBP, Val 574-Ser 649, accession AF283812), Gal-SMRT (human SMRT, Arg 1109, Gly 1330, accession U37146) and Gal-NCoR (mouse NCoR, Arg 2065-Gly 2287, accession U35312). VP16 fusions contained the 78 amino acid Herpes virus VP16 transactivation domain (Ala 413-Gly 490, accession X03141) fused to the N-terminus of the following proteins: VP-SXR (full-length, human SXR, accession AF061056). βgal contained the E. coli β-galactosidase coding sequences derived from pCH110 (accession U02445). Luciferase reporter constructs (TK-luc) contained the Herpes virus thymidine kinase promoter (−105/+51) linked to the indicated number of copies of the following response elements: CYP3A4x3(5′-TAGAATATGAACTCAAAGGAGGTCAGTGAGTGG-3′; SEQ ID NO:1), UAS_{G}x4(5′-CGACGGAGTACTGTCCTCCGTCG-3′; SEQ ID NO:2) and LXREx3. Wang et al., Mol. Cell 3:543-553, 1999. Docetaxel was obtained from Rhone-Poulenc Rorer (Collegeville, Pa.); 3′-p-hydroxypaclitaxel and 6α-hydroxypaclitaxel from Gentest (Woburn, Mass.); rifampicin, pregnenolone-16α-carbonitrile and paclitaxel were obtained from Sigma Chemical (St. Louis, Mo.) and ET-743 was obtained from the National Cancer Institute Drug Synthesis and Chemistry Branch.

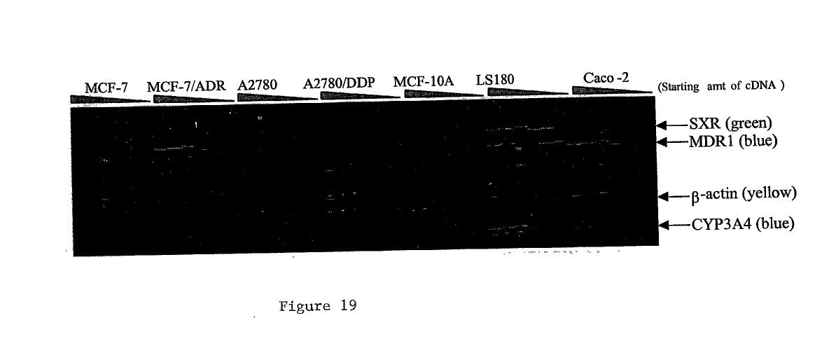

Given the expression patterns of SXR, MDR1, and CYP3A4 in normal tissues, it is reasonable that the mRNA for all three genes were present in LS180 and Caco-2 colon carcinoma cell lines. The data presented in FIG. 19 showing the induction of MDR1 and CYP3A4 expression in human LS180 cells by known activators of SXR are consistent with a role for SXR in this induction. Furthermore, our results demonstrating that SXR mRNA was present in MCF-7 cells is consistent with previously published data showing that SXR is expressed in human breast tumors. Moreover, we found that the expression of SXR and MDR1 was higher in the doxorubicin-resistant MCF-7/ADR cells. It is intriguing to speculate that these cells may have developed resistance in part due to induction of MDR1 expression in response to SXR ligands, and possibly that SXR is involved in the continued resistance of these cells in the presence of drug.

As a result, SXR is a target for the discovery of new drugs which modify expression of CYP2C8 and MDR1. For example, agents that are found to repress SXR can be combined with drugs that are known to be metabolized in the liver and/or cleared by biliary excretion in order to slow down the rate of drug elimination from the body. Moreover, co-administration of an SXR repressor may greatly improve the oral bioavailability of drugs by down-regulating CYP3A4 and MDR1 in the intestine. Therefore, as the “master” regulator of drug elimination, the activity of SXR can be manipulated to achieve a desired therapeutic effect. By down-regulating SXR, we will inhibit transient ligand-dependent increases in MDR1 AND CYP3A4 expression and enhance drug sensitivity.

Use of a standard model heterologous cell system to reconstitute SXR agonist and antagonist responsiveness allows SXR activity to be monitored in the absence of the metabolic events which may obscure the process being tested. Any suitable heterologous cell system may be used to test the activation of potential or known SXR nuclear receptor ligands, as long as the cells are capable of being transiently transfected with the appropriate DNA which expresses receptors, reporter genes, response elements, and the like. Cells which constitutively express one or more of the necessary genes may be used as well. Cell systems that are suitable for the transient expression of mammalian genes and which are amenable to maintenance in culture are well known to those skilled in the art. To test the activation of SXR by a variety of potential ligands, CV-1 cells may be transiently transfected with expression vectors for the appropriate receptors along with appropriate reporter constructs according to methods known in the art. Suitable reporter gene constructs are well known to skilled workers in the fields of biochemistry and molecular biology. Activity of the reporter gene can be conveniently normalized to the internal control and the data plotted as fold activation relative to untreated cells.

Any response element compatible with the assay system may be used. Oligonucleotide sequences which are substantially homologous to the DNA binding region to which the nuclear receptor binds are contemplated for use with the inventive methods. Substantially homologous sequences (probes) are sequences which bind the ligand activated receptor under the conditions of the assay. Response elements can be modified by methods known in the art to increase or decrease the binding of the response element to the nuclear receptor.

Coactivator recruitment assays have become established as a reliable method to identify and test the activity of nuclear receptor ligands (Blumberg et al., Genes Dev., 12:1269-1277 (1998); Forman et al., Nature, 395:612-615 (1998); Kliewer et al., Cell, 92:73-82 (1998); Krey et al., Mol. Endocrinol., 11:779-791 (1997)). In accordance with the present invention, a mammalian two-hybrid coactivator recruitment assay was developed to examine whether putative ligands could promote a functional association between SXR and a coactivator as a test of a ligand's ability to modify the transcription of genes regulated by the SXR.

For in vitro assays, after addition of the putative ligand to the mixture of components describe above and mixing, the mixture is incubated under conditions such that coactivator may be recruited. The formation of complexes in the mixture are analyzed by electrophoretic mobility shift (gel shift assay), however, any method of measuring complex formation may be used. Techniques such as, for example, fluorescence-resonance energy transfer, scintillation proximity assays, luminescence proximity assays and the like are suitable, however those of skill in the art are capable of using any number of methods to measure complex formation.

Strategies to downregulate SXR expression include stable transfection of the full length antisense SXR and transfection with antisense oligonucleotides positioned at various points along the SXR coding sequence or transfection of cells with a dominant negative version of SXR to block the activity SXR protein. A dominant negative version of SXR may be created by truncating the protein at the binding domain or making C-terminal truncations deleting only the C-terminal transactivation domain.

The invention is further described and illustrated in the following examples, which are not intended to be limiting.

### EXAMPLES

### Example 1

### Paclitaxel Activates SXR

To explore whether paclitaxel can activate SXR, CV-1 cells were transiently transfected with vectors expressing Gal4 fused to the ligand binding domain of human SXR (Gal-L-SXR) or to the human RXRα ligand binding domain (Gal-L-RXR). After transfection, cells were treated with the following compounds: 10 μM rifampicin, 10 μM SR12813, 10 μM pregnenolone-16α-carbonitrile (Preg-16-CN), 10 μM paclitaxel, 100 nM LG268, 10 μM 6α-hydroxypaclitaxel and 10 μM 3′p-hydroxypaclitaxel. The Gal4 reporter activity was normalized to the internal β-galactosidase control and the data plotted as fold activation relative to untreated cells. All transfections contained the Gal4 reporter and a β-galactosidase expression vector as an internal control.

CV-1 cells were grown in Dulbecco's Modified Eagle's medium supplemented with 10% resin-charcoal stripped fetal bovine serum, 50 U/ml penicillin G and 50 μg/ml streptomycin sulfate (DMEM-FBS) at 37° C. in 5% CO₂. One day prior to transfection, cells were plated to 50-80% confluence using phenol-red free DMEM-FBS. Cells were transiently transfected by lipofection according to prior art methods. Wang et al., Mol. Cell 3:543-553, 1999. Reporter constructs (300 ng/10⁵ cells), cytomegalovirus driven expression vectors (25 ng/10⁵ cells) were added as indicated along with βgal (500 ng/10⁵ cells) as an internal control. After two hours, the liposomes were removed and replaced with fresh media. Cells were treated for approximately 24 hours with phenol-red free DMEM-FBS containing the indicated compounds. After exposure to ligand, the cells were harvested and assayed for β-galactosidase activity according to standard methods. The potential cytotoxic effects of paclitaxel, docetaxel and ET-743 were minimal when used at the indicated concentrations and treatment times.

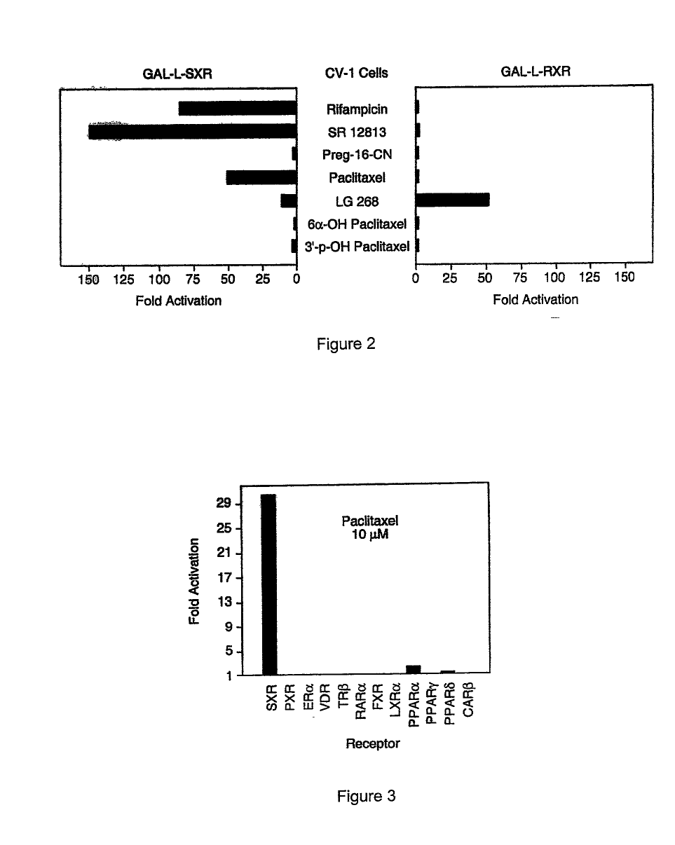

The Gal-L-SXR chimeric receptor was activated by 10 pM doses of the SXR agonists rifampicin and SR12813, but not by pregnenolone-16α-carbonitrile, a specific agonist of the mouse ortholog of SXR. Paclitaxel strongly activated SXR (50-fold) at clinically-relevant concentrations (EC₅₀≈5 μM). See FIG. 2. Forman et al., Nature 395:612-615, 1998; Forman et al., Proc. Natl. Acad. Sci. USA 94:4312-4317, 1997; Forman et al., Cell 83:803-812, 1995; Forman et al., Cell 81:541-550, 1995. No activation was seen with the RXR ligand LG268 (100 nM) or with 3′-p-hydroxypaclitaxel or 6a-hydroxypaclitaxel, the products of paclitaxel metabolism by CYP3A4 and CYP2C8, respectively. See FIG. 2. Qualitatively similar results were seen with the wild-type SXR.

To test whether paclitaxel specifically activates SXR, transfections were performed as above using previously described plasmids. As positive controls, each receptor was activated by it cognate ligand as follows: mouse PXR (23-fold, 10 μM Preg-16-CN), human ERα (15-fold, 100 nM 17β-estradiol), human VDR (59-fold, 100 nM, 1,25-dihydroxyvitmin D₃) human TRβ (19-fold, 100 nM triiodothyronine), human RARα (315-fold, 100 nM Am580), human LXRα (4.5-fold, 30 μM hyodeoxycholic acid methyl ester), mouse PPARα (13-fold, 5 μM Wy 14,643), mouse PPARγ (20-fold, 1 μM rosiglitazone), mouse PPARδ (14-fold, 1 μM arbaprostacyclin), mouse CARβ (50-fold repression 5 μM androstanol). After exposure to ligand, the cells were harvested and assayed for luciferase and βgal according to known methods. Activation of SXR by paclitaxel was specific to SXR since it had no effect on RXR, the heterodimeric partner of SXR, or other nuclear receptors including PXR (the mouse ortholog of SXR), estrogen receptor α (ERα), vitamin D receptor (VDR), thyroid hormone receptor β (TRβ), retinoic acid receptor α (RARα), FXR, LXRα, PPARα, PPARγ, PPARδ and CARβ. See FIG. 3.

### Example 2

### SXR Induces CYP2C8 and MDR1 Expression

To compare paclitaxel's ability to activate CYP3A4 expression with that of other SXR agonists, primary human hepatocytes which natively express SXR, prepared according to known methods, were treated with SXR agonists and CYP3A4 expression was monitored by northern analysis. Northern analysis was performed as follows. Primary human hepatocytes were obtained from Clonetics (Walkersville, Md.) and maintained in Hepatocyte Maintenance Medium supplemented with dexamethasone and insulin according to the vendors instructions. Cells were treated with the indicated SXR agonists for 48 hours and total RNA was isolated using the Trizol reagent.

Human LS180 cells were maintained in Eagle's minimal essential medium supplemented with 10% fetal bovine serum, 1 mM sodium pyruvate, 2 mM L-glutamine, non-essential amino acids, 50 U/ml penicillin G and 50 μg/ml streptomycin sulfate. One day prior to treatment, the LS180 cells were switched to phenol-red free media containing 10% resin-charcoal stripped fetal bovine serum and then treated for an additional 24 hours with the indicated compounds. Northern blots were prepared from total RNA and analyzed with the following probes: MDR1 (accession NM_{—}000927, nucleotides 843-1111), CYP2C8 (accession NM_{—}000770, nucleotides 700-888), CYP3A4 (accession M18907, nucleotides 1521-2058), RXRα (accession X52773, nucleotides 738-1802) and GAPDH (accession NM_{—}002046, nucleotides 101-331). Note that the CYP2C8 probe was specific as it did not cross-hybridize to the two most closely related members of the CYP2C family; CYP2C9 and CYP2C19 (data not shown).

For transfection of human LS180 cells, VP-SXR and/or GFP (Topaz variant, Packard) were transfected with lipofectamine (GibcoBRL) according to the manufacturer's instructions. Cells were transfected and maintained in phenol-red free media containing 10% resin-charcoal stripped fetal bovine serum. After 48 hours, cells were sorted on a MoFlo (Cytomation, Fort Collins, Colo.) flow cytometer. Data was acquired using dual laser excitation. Scatter signals were acquired with a HeNe laser 633 nm (Spectra-Physics, Mountain View, Calif.). All fluorescence excitation was done at 488 nm from an Innova-90 Argon laser (Coherent, Santa Clara, Calif.) at 500 mW. GFP emission was measured through a 530DF30 filter (Omega Optical, Brattleboro, Vt.). GFP positive cells were sorted using 60 psi, 94,000 kHz droplet formation with a 70-micron nozzle at a flow rate of 12,000/second. Total RNA was prepared from transfected (GFP-positive) cells and analyzed as above. Each experiment was repeated three or more times with similar results. The potential cytotoxic effects of paclitaxel, docetaxel and ET-743 were minimal when used at the indicated concentrations and treatment times. For primary human hepatocytes, each experiment was performed using cells obtained from different donors.

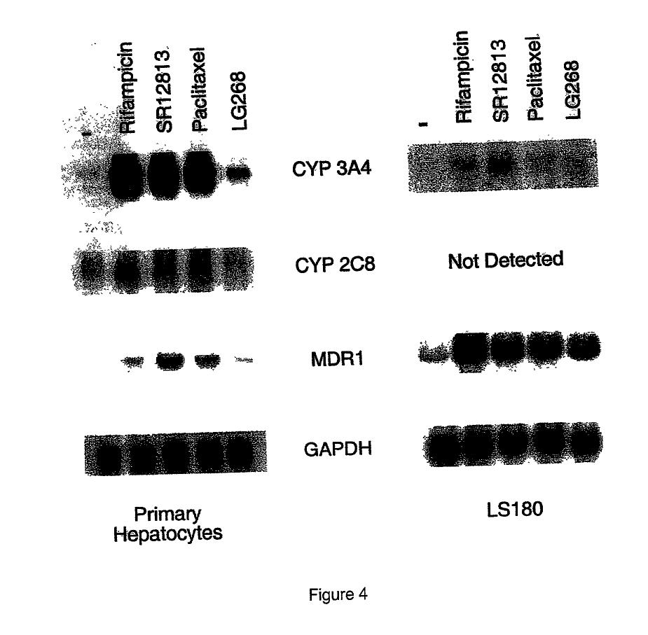

Primary human hepatocytes (left panel) were treated for 48 hours and human LS180 cells (right panel) were treated for 24 hours with control media or media supplemented with the following compounds: 10 μM rifampicin, 10 μM SR12813, 10 μM paclitaxel or 100 nM LG268. Total RNA was prepared and northern blots were probed with CYP3A4, CYP2C8, MDR1 and a GADPH control (glyceraldehyde-3-phosphate dehydrogenase) as indicated. See FIG. 4. Consistent with the transfection experiments (FIG. 2), rifampicin, SR12813 and paclitaxel and other SXR agonists induced expression of CYP2C8, the other cytochrome P450 enzyme that inactivates paclitaxel in vivo. Note that CYP2C8 expression was not detected in the LS180 cells. Rifampicin, paclitaxel (FIG. 4, left panel) and hyperforin (data not shown) strongly activated CYP2C8 expression, whereas the RXR ligand LG268 was inactive. The fold response to SR12813 was less than that seen with other SXR agonists and varied from one hepatocyte donor to another (FIG. 4, left panel and data not shown). Activation by rifampicin, paclitaxel and hyperforin suggests that human CYP2C8 is a downstream target of SXR activation. Since SXR agonists induced expression of enzymes required for paclitaxel degradation, SXR regulation MDR1 (P-glycoprotein) was also tested. In primary human hepatocyte cultures, the expression of MDR1 was enhanced by several SXR agonists (FIG. 4, left panel). In intestinal cells (LS180 colon cancer cells), CYP3A4, which is expressed at low levels in intestinal cells, was induced by SXR ligands (FIG. 4, right panel). Similarly, MDR1 was very strongly induced by the same SXR ligands (FIG. 4, right panel) as well as by hyperforin (data not shown), another potent SXR ligand. These pharmacologic data strongly suggest that MDR1 is an SXR target gene in both the intestine and liver.

### Example 3

### Activation of MDR1 by a Constitutively Active SXR

To further confirm the link between SXR and MDR1, a constitutively active variant of SXR was assayed for MDR1 activation in the absence of SXR ligands. CV-1 cells were sorted using 60 psi, 94,000 kHz droplet formation with a 70-micron nozzle at a flow rate of 12,000/second. Total RNA was prepared from transfected (GFP-positive) cells and analyzed as above. Each experiment was repeated three or more times with similar results. The potential cytotoxic effects of paclitaxel, docetaxel and ET-743 were minimal when used at the indicated concentrations and treatment times. For primary human hepatocytes, each experiment was performed using cells obtained from different donors.

Primary human hepatocytes (left panel) were treated for 48 hours and human LS180 cells (right panel) were treated for 24 hours with control media or media supplemented with the following compounds: 10 μM rifampicin, 10 μM SR12813, 10 μM paclitaxel or 100 nM LG268. Total RNA was prepared and northern blots were probed with CYP3A4, CYP2C8, MDR1 and a GADPH control (glyceraldehyde-3-phosphate dehydrogenase) as indicated. See FIG. 4. Consistent with the transfection experiments (FIG. 2), rifampicin, SR12813 and paclitaxel and other SXR agonists induced expression of CYP2C8, the other cytochrome P450 enzyme that inactivates paclitaxel in vivo. Note that CYP2C8 expression was not detected in the LS180 cells. Rifampicin, paclitaxel (FIG. 4, left panel) and hyperforin (data not shown) strongly activated CYP2C8 expression, whereas the RXR ligand LG268 was inactive. The fold response to SR12813 was less than that seen with other SXR agonists and varied from one hepatocyte donor to another (FIG. 4, left panel and data not shown). Activation by rifampicin, paclitaxel and hyperforin suggests that human CYP2C8 is a downstream target of SXR activation. Since SXR agonists induced expression of enzymes required for paclitaxel degradation, SXR regulation MDR1 (P-glycoprotein) was also tested. In primary human hepatocyte cultures, the expression of MDR1 was enhanced by several SXR agonists (FIG. 4, left panel). In intestinal cells (LS180 colon cancer cells), CYP3A4, which is expressed at low levels in intestinal cells, was induced by SXR ligands (FIG. 4, right panel). Similarly, MDR1 was very strongly induced by the same SXR ligands (FIG. 4, right panel) as well as by hyperforin (data not shown), another potent SXR ligand. These pharmacologic data strongly suggest that MDR1 is an SXR target gene in both the intestine and liver.

### Example 3

### Activation of MDR1 by a Constitutively Active SXR

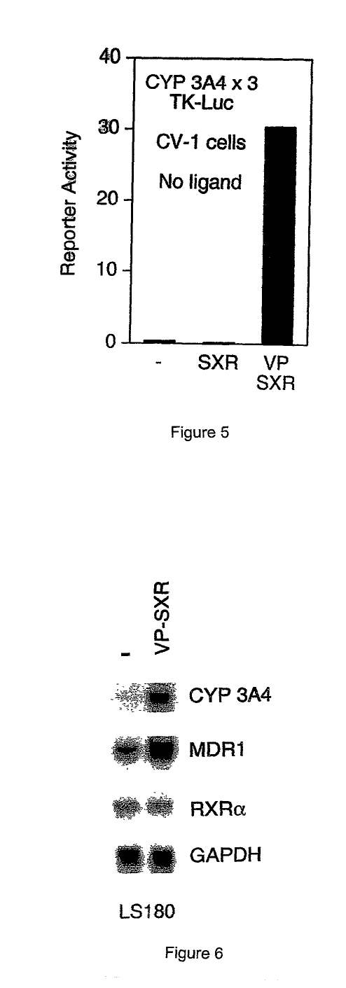

To further confirm the link between SXR and MDR1, a constitutively active variant of SXR was assayed for MDR1 activation in the absence of SXR ligands. CV-1 cells were transiently transfected as described in Example 1 with an SXR reporter (CYP3A4x3-TK-luc) and expression vectors for native human SXR or human SXR fused to the Herpes VP16 transactivation domain (VP-SXR), a constitutively active version of SXR. After transfection, cells were maintained in media without an SXR agonist. Reporter activity was determined and normalized to the internal β-galactosidase control. As expected, wild-type SXR was inactive in the absence of ligand, however the VP-SXR chimera constitutively activated a reporter construct containing SXR response elements from the CYP3A4 promoter. See FIG. 5.

human LS180 cells were transiently transfected with a green fluorescent protein (GFP) expression vector alone (−) or with GFP and VP-SXR and maintained in media lacking SXR agonists to determine whether the constitutively active SXR activates endogenous CYP3A4 and MDR1 expression. Cells were harvested 48 hours after transfection and transfected cells (i.e., those expressing GFP) were collected by flow cytometry and analyzed by northern analysis as described in Example 2 above. In the absence of ligand, VP-SXR induced expression of CYP3A4 and MDR1 but had little effect on the RXRα and GAPDH control transcripts (FIG. 6). The effect of VP-SXR was specific: VP-FXR, a chimera with another nuclear receptor, was inactive, as was a VP-SXR construct that lacked the SXR DNA binding domain (data not shown). Taken together, these data demonstrate that SXR regulates MDR1 expression in the intestine.

### Example 4

### Chemical Modifications Dissociate the Antineoplastic and Xenobiotic Clearance Activates of Paclitaxel

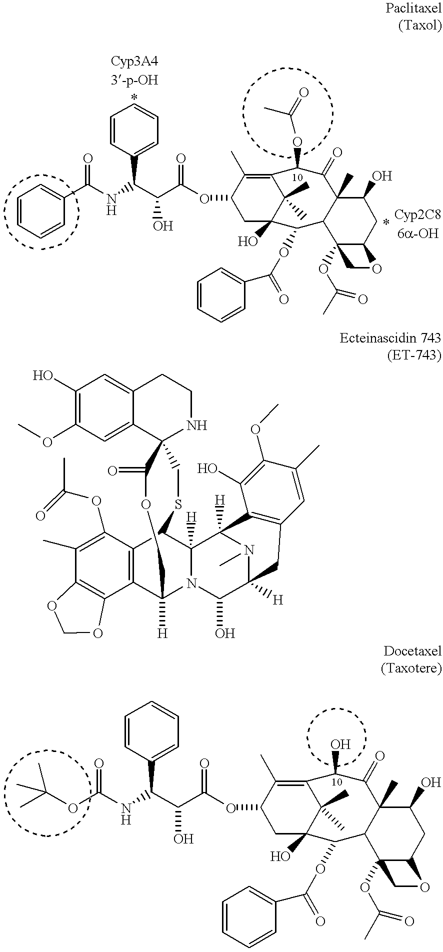

The transcriptional effects of docetaxel (taxotere), a clinically-tested paclitaxel analog with similar antineoplastic activity, was compared with paclitaxel. Docetaxel possesses a hydroxyl group in place of the acetyl moiety at position 10 and an N-tert-butoxycarbonyl group instead of the N-benzoyl group on the terminal side chain. These regions are highlighted with dotted circles. The positions where paclitaxel is hydroxylated by CYP3A4 and CYP2C8 are also indicated. See structure I (paclitaxel), structure II (docetaxel) and structure III (ecteinascidin 743; ET-743), above. These structural differences have little effect on antineoplastic potency. Both taxanes inhibit microtubule depolymerization at similar concentrations.

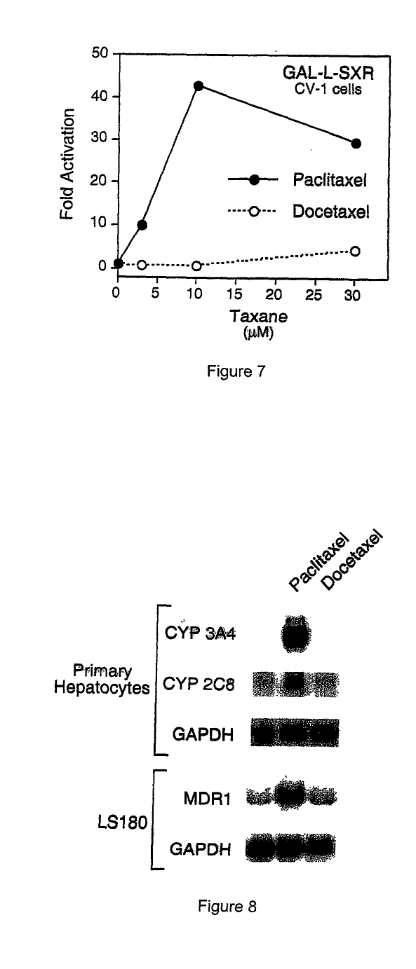

In contrast, these differences are critical to SXR responsiveness. After transfection with Gal-L-SXR as in Example 1, cells were treated with the indicated concentrations of paclitaxel or docetaxel and fold activation of the Gal-L-SXR reporter was assayed. Docetaxel did not effectively activate Gal-L-SXR at any concentration tested (FIG. 7). Thus, the cytotoxic effects of the taxanes are dissociated from their SXR-mediated transcriptional effects. To confirm this, docetaxel was assayed for activation of endogenous SXR-target genes. Primary human hepatocytes (upper panel) and human LS180 cells (lower panel) were treated as in Example 2 with control media or media supplemented with 10 μM paclitaxel or 10 μM docetaxel. Total RNA was prepared and northern blots were probed with CYP3A4, CYP2C8, MDR1 and a GADPH control.

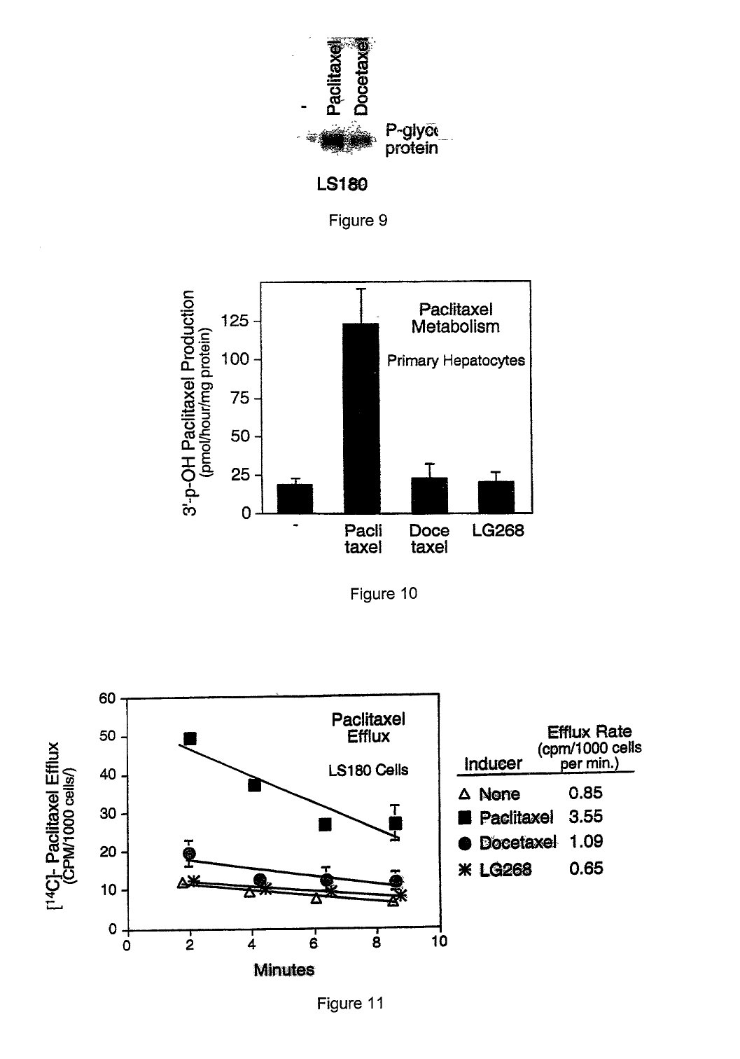

Docetaxel failed to activate CYP3A4 and CYP2C8 mRNA expression in primary human hepatocytes and did not induce MDR1 expression in LS180 human intestinal cells. See FIG. 8. Similarly, western analysis using a P-glycoprotein antibody of LS180 human cells treated with control media or media supplemented with 10 μM paclitaxel or 10 μM docetaxel for 48 hours indicated that paclitaxel was much more effective than docetaxel in inducing MDR1 protein (P-glycoprotein) expression in LS180 human cells (FIG. 9).

Western Blotting was performed according to the following methods. Human LS180 cells in log phase growth were treated for 48 hours with the compounds indicated in the pertinent Figures. The cells were harvested, washed with phosphate buffered saline (PBS) and homogenized using 12-15 strokes of a Wheaton teflon-glass homogenizer. Cell debris was removed by centrifugation at 1500×g for 10 minutes, and the resulting supernatant was sedimented at 150,000×g for one hour at 4° C. to pellet the membranes. The membrane pellets were resuspended in PBS containing 1 mM phenylmethylsulfonyl fluoride and protein concentrations were determined according to standard prior art methods. Protein extracts (20 μg/lane) were separated on a 4-15% gradient SDS polyacrylamide gel and transferred electrophoretically to PVDF membranes. The membranes were blocked with 5% non-fat dry milk in PBS with 0.1% Tween-20 (PBS-T) before incubation with a 1:500 dilution of P-glycoprotein antibody (Ab-1, Oncogene Research Products, Boston, Mass.) in blocking buffer for six hours at room temperature. Following several washes with PBS-T, membranes were incubated with a 1:1000 dilution of horseradish peroxidase-conjugated secondary anti-rabbit IgG antibodies. (Santa Cruz Biotechnology, Santa Cruz, Calif.) in blocking buffer for one hour at room temperature. Immunoblot detection was performed using the ECL detection system under conditions suggested by the manufacturer (Amersham).

### Example 5

### Docetaxel does not regulate Paclitaxel Metabolism and Efflux

To test the ability of docetaxel to regulate drug clearance, paclitaxel metabolism and efflux induction by taxane analogs was assayed. Primary human hepatocytes were maintained in control media or media supplemented with 10 μM paclitaxel, 10 μM docetaxel or 100 nM LG268. After this induction period, the antineoplastic agents were removed and CYP3A4 activity (formation of paclitaxel hydroxylase) was measured as follows using paclitaxel as a substrate for the production of 3′-p-hydroxylpaclitaxel. Error bars indicate the standard deviation of triplicate data points. The entire experiment was repeated twice with similar results.

Primary human hepatocytes were treated with the indicated drugs (10 μM paclitaxel, 10 μM docetaxel, 100 nM LG268) for 48 hours to allow for accumulation of SXR-induced proteins. Following this induction period, cells were washed and incubated for an additional one hour in fresh hepatocyte maintenance media to allow for efflux of intracellular drug. This step effectively removed the inducer as the levels of paclitaxel and its metabolites measured in the media following this one hour wash step was less than 6% of the final amounts determined from CYP3A4 activity. Fresh media containing 10 μM paclitaxel were then added for an additional three hours. After three hours, the media were collected and the concentrations of 3′-p-hydroxypaclitaxel in the media was determined by HPLC. Following the assays, hepatocytes from each well were collected and the protein content was determined using the Bradford assay. Results were normalized to pmol of 3′-p-hydroxypaclitaxel formed per hour per mg protein. The entire experiment was repeated twice with cells derived from different donors and yielded similar results. Whereas paclitaxel pretreatment induced an approximate 5-fold increase in the rate of 3′-p-hydroxypaclitaxel production, both docetaxel and the control RXR ligand (LG268) had no effect on CPY3A4 activity. See FIG. 10.

Taxane-induced drug efflux was measured using pretreated LS180 human colon cancer cells. The rate of drug efflux was measured. LS180 human cells were induced for 48 hours with 10 μM paclitaxel, 10 μM docetaxel or 100 nM LG268 as indicated. After induction, cells were loaded with [¹⁴C]-paclitaxel for 15 minutes and the rate of paclitaxel efflux was determined by measuring the release of [¹⁴C]-paclitaxel from cells at multiple time points. Individual data points are the means of triplicate determinations, error bars represent standard deviation and the lines are lines of regression. The slope of each line (rate of efflux) was compared to the slope obtained in the control (untreated) cells using an analysis of covariance. The rate of drug efflux from paclitaxel pretreated cells was significantly faster than that from untreated cells (P=−0.002), while the rate of efflux from docetaxel (P=0.366) and LG268 (P=0.094) pretreated cells did not differ from controls. The entire experiment was performed three times with similar results. Following a 48 hour induction with the indicated drugs (10 μM paclitaxel, 10 μM docetaxel, 100 nM LG268), LS180 human cells were washed and incubated for an additional one hour in fresh media to allow for efflux of intracellular drug. The cells were then incubated in media supplemented with 10 μM [¹⁴C]-paclitaxel (4.9 pCi/pmol, Moravek Biochemicals, Brea, Calif.) for 15 minutes. The uptake of ¹⁴C-paclitaxel reached maximum levels at 10-12 minutes (data not shown). After 15 minutes, the cells were then rapidly centrifuged through silicone oil to remove all traces of extracellular radioactivity, resuspended in fresh media, and cell counts determined. At multiple time points over the next 10 minutes, triplicate aliquots of the cell suspension (approx. 1×10 cells/aliquot) were again centrifuged through silicon oil and the radioactivity in the cell pellet measured by quench-corrected liquid scintillation counting. The rate of [¹⁴C]-paclitaxel efflux was determined as the slope of the [¹⁴C]-paclitaxel versus time plots using all data. The slope for each inducer was compared to the slope obtained in the control (untreated) cells using an analysis of covariance. The entire experiment was repeated three times with cells derived from different donors and yielded similar results. See FIG. 11.

As predicted, the rate of drug efflux from paclitaxel treated cells was significantly greater than that from untreated or docetaxel treated cells. Taken together, these data demonstrate that SXR activation can be used as a tool to identify drug analogs that do not induce hepatic metabolism or P-glycoprotein mediated drug transport.

### Example 6

### Docetaxel Fails to Displace Nuclear Receptor Corepressors from SXR

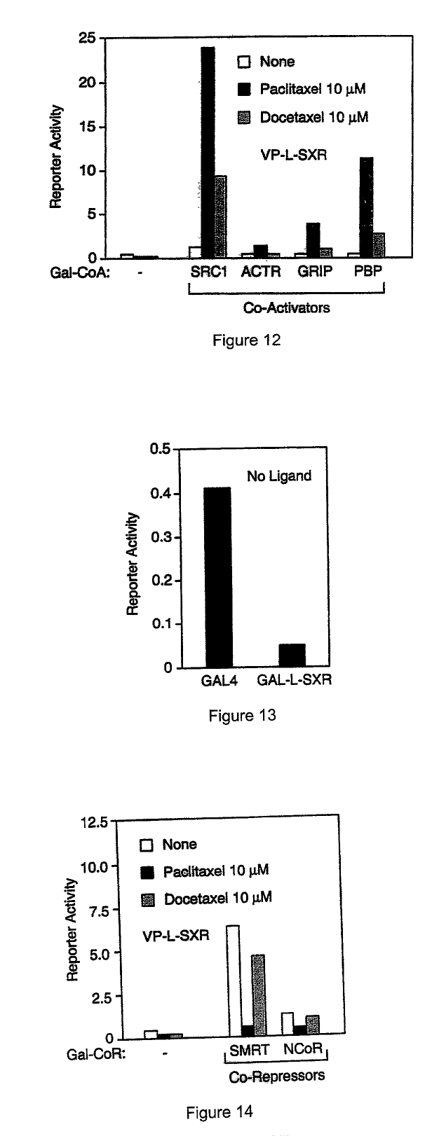

A mammalian two-hybrid assay was used to compare the effects of paclitaxel and docetaxel on coregulator recruitment. CV-1 cells were transiently transfected as in Example 1 with a Gal4 reporter and an expression vector containing the VP16 transactivation domain linked to the ligand binding domain of SXR (VP-L-SXR). In addition, cells were also transfected with expression vectors for the Gal4 DNA binding domain (−) or Gal4 linked to the receptor interaction domains of the nuclear receptor coactivators SRC1, ACTR, GRIP or PBP, as indicated. After transfection, cells were treated with control media or media containing 10 μM paclitaxel or 10 μM docetaxel. In this system, reporter expression is activated if VP16 becomes tethered to the promoter via an SXR coactivator interaction. See Wang et al., Mol. Cell 3:543-553, 1999, the disclosures of which are hereby incorporated by reference. As expected, treatment of cells with either paclitaxel or docetaxel did not promote an interaction between SXR and the control Gal4 DNA binding domain. See FIG. 12. However, paclitaxel did promote an interaction with all of the coactivators tested except CBP (FIG. 12 and data not shown). The hierarchy of the interaction was SRC1>PBP>GRIP>ACTR. Docetaxel promoted a qualitatively similar response, though its effect was 25-40% less than that seen with paclitaxel. These findings indicate that docetaxel has the potential to act as a partial SXR agonist, however, this partial response cannot fully account for docetaxel's crippled activity on SXR.

### Example 7

### SXR-Corepressor Interactions

The diminished response to docetaxel could reflect altered corepressor displacement. To explore the possibility that corepressors play a role in SXR action, SXR repression of basal transcription was tested. CV-1 cells were transiently transfected with the Gal4 DNA binding domain or Gal-L-SXR. Reporter activity was measured in cells maintained in the absence of ligand. Unliganded Gal-L-SXR repressed basal transcription by about 4-fold. See FIG. 13.

A mammalian two-hybrid assay was used to evaluate potential SXR-corepressor interactions. CV-1 cells were transiently transfected as in Example 6, but the Gal-coactivator expression vectors were replaced with expression vectors for Gal4 linked to the receptor interaction domains of the nuclear receptor corepressors SMRT or NCoR, as indicated. After transfection cells were treated with control media or media containing 10 μM paclitaxel or 10 μM docetaxel. As shown in FIG. 14, unliganded SXR interacted with the nuclear corepressor SMRT. More importantly, paclitaxel reversed this interaction whereas docetaxel had little effect. The SXR-NCOR interaction was significantly weaker, though the differential response of the two drugs was maintained. These data indicate that the restricted activity of docetaxel on SXR is closely related to its inability to displace corepressors.

### Example 8

### Ecteinascidin-743 Antagonizes SXR Action

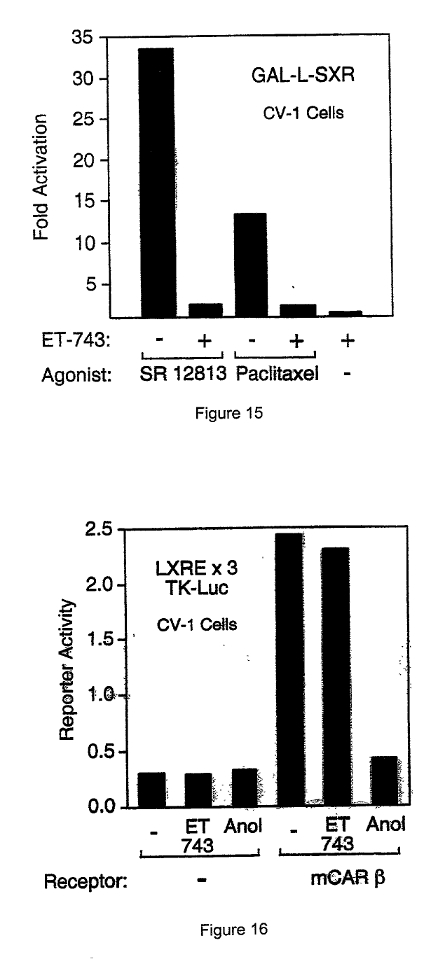

CV-1 cells were transiently transfected with as in Example 1 with Gal-L-SXR. After transfection, cells were treated with 10 μM SR12813, 10 μM paclitaxel and/or 50 nM ET-743, as indicated in FIG. 15. ET-743 (50 nM) was extremely potent and effective inhibitor of SR12813- and paclitaxel-induced activation of Gal-L-SXR (FIG. 15). In contrast, ET-743 had no effect on the transcriptional activity of CARβ, a constitutively active nuclear receptor whose transcription is suppressed by androstanol and whose ligand-responsiveness overlaps that of SXR.

CV-1 cells were transfected with an LXREx3-TK-luc reporter and an expression vector for CARβ, where indicated in FIG. 16. After transfection, cells were treated with control media (−) or media containing 5 μM androstanol or 50 nM ET-743. CARβ was transcriptionally active in the absence of ligand and is inhibited by androstanol, Forman et al., Nature 395:612-615, 1998, but not ET-743. See FIG. 16.

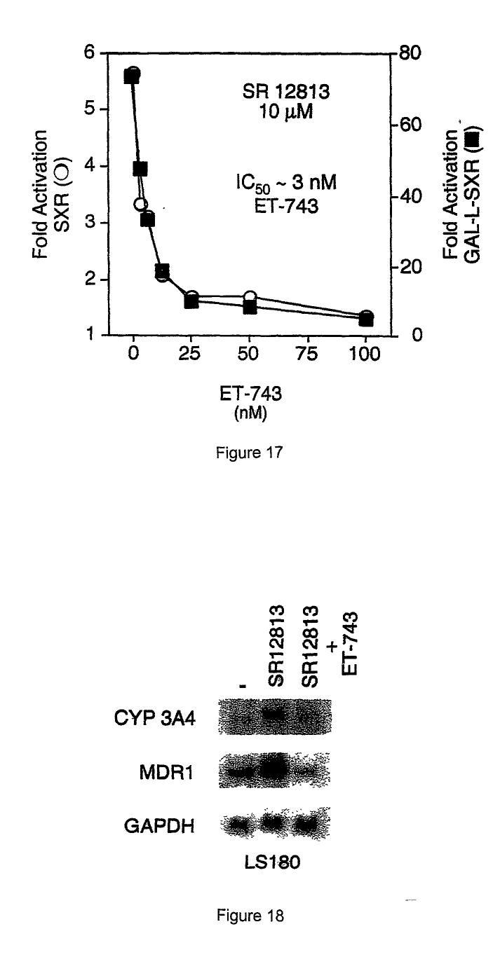

Dose response studies demonstrated that ET-743 maximally inhibited both wild-type and Gal-L-SXR at concentrations of 25-50 nM; half-maximal inhibition (IC₅₀) was observed at approximately 3 nM (FIG. 17). CV-1 cells were transiently transfected with SXR and a CYP3A4x3 TK-luc reporter or with Gal-L-SXR and UAS_{G}x4 TK-luc. After transfection, cells were treated with control media, media supplemented with 10 μM SR12813 or 10 μM SR12813 and the indicated concentrations of ET-743. Fold activation was determined and plotted relative to untreated cells. This dose-response profile matches the reported inhibition of trichostatin-induced MDR1 transcription and antineoplastic effects of ET-743. Izbicka et al., Ann. Oncol. 10:1233-1240, 1999; Martinez et al., Proc. Natl. Acad. Sci. USA 96:3496-3501, 1999; Minuzzo et al., Proc. Natl. Acad. Sci. USA 97:6780-6784, 2000; Jin et al., Proc. Natl. Acad. Sci. USA 97:6775-6779, 2000. Northern analysis indicated that ET-743 (40 nM) effectively inhibited SR12813-induced activation of both CYP3A4 and MDR1 but had no effect on the GAPDH control (FIG. 18). LS180 cells were treated for 16 hours with control media or media supplemented with 10 μM SR12813±40 nM ET-743. Total RNA was prepared and northern blots were probed as in Example 2. Taken together, these data suggest that ET-743 represses MDR1 transcription by antagonizing SXR.

### Example 9

### Basal expression of SXR, CYP3A4, and MDR1 in human tumor cells

TABLE I
Basal Expression of SXR, MDR1 and CYP3A4

| | SXR | MDR1 | CYR3A4 |
|---|---|---|---|
| MCF-7 | +/− | − | − |
| MCF-7/ADR | + | ++ | − |
| MCF-10A | − | − | − |
| A2780 | − | − | − |
| A2780/DDP | − | − | + |
| OVCAR-3 | − | +/− | − |
| LS18O | +++ | + | +++ |
| Caco-2 | +/− | ++ | + |

Expression numbers were first calculated by dividing the slope for the gene of interest by the slope for β-actin and multiplied by 1000.[66]. Numbers were then applied to the following scale: (−) = undetectable; (+/−) = 0.01-1.0; (+) = 1.1-10.0; (++) = 10.1-100; (+++) = 100.1-1000.

Because little is known about the expression of SXR in human tumors, a RT-PCR assay for the simultaneous and semi-quantitative detection of SXR, MDR1 and CYP3A4 mRNA was developed, based on the methods of Luehrsen et al., Biotechniques 22:168-174, 1997 and Johnston et al., Canc. Res. 55:1407-1412, 1995. The method involves isolation of mRNA from frozen tissues or from cultured cell lines, reverse transcription of the mRNA to the corresponding cDNA, PCR amplification of serial dilutions of cDNA using 5′-fluorescent tagged primers, and separation of labeled fragments on an ABI Prism 377 DNA Sequencer. mRNA was isolated from cells using RNAzol B, and then reverse transcribed into cDNA. PCR was performed using increasing dilutions of cDNA and 5′-fluorescently-tagged primers. PCR reactions were run separately under optimal conditions for amplification and the reactions are pooled and run on the same sequencing gel for quantitation an ABI Prism 377 sequencer. The expression level of the various genes is then quantified using GeneScan software (Version 3.1). Size standards (red bands) are included in every lane. Other bands on the gel represent genes irrelevant to our study that were included in the analysis. Individual gene expression is calculated from the linear portion of the dilution versus PCR product curves normalized to the expression of α-actin [66]. Finally, the numbers are used to assign expression levels according to the following scale: (−)=Undetectable; (+/−)=0.01-1.0; (+)=1.1-10.0; (++)=10.1-100; (+++)=100.1-1000.

A representative sequencing polyacrylamide gel is shown in FIG. 19. As depicted in the Figure, the gene fragments for SXR, MDR1, and CYP3A4 can been seen in LS180 human cells at their appropriate locations on the gel compared to the size standards. Using this method, the expression of SXR, MDR1 and CYP3A4 was determined in a panel of human tumor cell lines. See FIG. 19. As shown in Table I above, SXR mRNA was detected in 4 of the 8 cell lines tested. Basal expression of SXR was detected in parental MCF-7 breast cancer cells, their doxorubicin-resistant variant MCR-7/ADR, and two colon carcinoma cell lines LS180 and Caco-2. The range of SXR mRNA expression was very wide, ranging from undetectable to the relatively high level found in LS180 human cells. Furthermore, only the human LS180 and Caco-2 cells expressed detectable levels of both MDR1 and CYP3A4 at baseline.

1. A method of modifying drug pharmacokinetics which comprises altering the activity of SXR on expression levels of CYP2C8 or MDR1.

2. A method of claim 1 wherein said alteration in SXR activity alters CYP2C8 expression.

3. A method of claim 1 wherein said alteration in SXR activity alters MDR1 expression.

4. A method of modifying multiple drug resistance which comprises altering SXR activity.

5. A method of claim 1 or 4 wherein drug catabolism is altered.

6. A method of claim 5 wherein drug catabolism is reduced.

7. A method of claim 5 wherein drug catabolism is increased.

8. A method of claim 1 or 4 wherein drug intestinal efflux is altered.

9. A method of claim 8 wherein drug intestinal efflux is reduced.

10. A method of claim 8 wherein drug intestinal efflux is increased.

11. A method of claim 1 or 4 wherein drug oral absorption is altered.

12. A method of claim 11 wherein drug oral absorption is reduced.

13. A method of claim 11 wherein drug oral absorption is increased.

14. A method of claim 1 or 4 wherein drug biliary excretion is altered.

15. A method of claim 14 wherein drug biliary excretion is reduced.

16. A method of claim 14 wherein drug biliary excretion is increased.

17. A method of claim 1 or 4 which comprises altering SXR mRNA levels.

18. A method of claim 1 or 4 which comprises altering SXR protein levels.

19. A method of claim 1 or 4 which comprises altering the ability of SXR to recruit coactivator.

20. A method of claim 1 or 4 which comprises altering the displacement of corepressor from SXR.

21. A method of claim 1 or 4 wherein said drug is a taxane.

22. A method of claim 1 or 4 which comprises administering an SXR antagonist.

23. A method of claim 22 wherein said SXR antagonist is ecteinascidin-743.

24. A method of claim 1 or 4 which comprises administering an SXR agonist.

25. A method of claim 1 or 4 which comprises administering a ribozyme which cleaves mRNA encoding SXR, an SXR coactivator or an SXR corepressor.

26. A method of claim 1 or 4 which comprises administering an antisense oligonucleotide which suppresses transcription or translation of SXR, an SXR coactivator or an SXR corepressor.

27. A method of identifying drugs with improved pharmacokinetic properties or activity which comprises screening drug candidates for their ability to modulate SXR.

28. A method of claim 27 which comprises identifying drugs having altered efflux characteristics by screening drug candidates for their ability to modulate the activity of SXR on expression levels of CYP2C8 or MDR1.

29. A method of claim 27 which comprises identifying drugs having altered catabolism by screening drug candidates for their ability to modulate the activity of SXR on expression levels of CYP2C8 or MDR1.

30. A method of claim 27 which comprises identifying drugs having altered oral bioavailability by screening drug candidates for their ability to modulate the activity of SXR on expression levels of CYP2C8 or MDR1.

31. A method of claim 27 which comprises identifying drugs having altered biliary excretion by screening drug candidates for their ability to modulate the activity of SXR on expression levels of CYP2C8 or MDR1.

32. A method of any of claims 27-31 wherein said drug candidates are taxanes.

33. A method of any of claims 27-32 which comprises monitoring SXR activity in cells in vivo or in vitro.

34. A method of claim 33 wherein said monitoring of SXR activity comprises monitoring the expression of an endogenous SXR regulated gene.

35. A method of claim 34 wherein said endogenous SXR regulated gene is a gene selected from the group consisting of CYP3A4, CYP2C8 and MDR1.

36. A method of claim 33 wherein said monitoring of SXR activity comprises monitoring the expression of a synthetic reporter gene under the control of control elements responsive to SXR.

37. A method of claim 33 wherein said monitoring of SXR activity comprises monitoring the expression of a chimeric gene, wherein the protein encoded by said chimeric gene maintains the ability to respond to SXR ligands.

38. A method of any of claims 27-31 which comprises monitoring SXR activity in cells in vitro.

39. A method of claim 38 wherein said monitoring of SXR activity comprises monitoring coactivator recruitment.

40. A method of claim 38 wherein said monitoring of SXR activity comprises monitoring corepressor displacement.

41. A method of claim 38 wherein said monitoring of SXR activity comprises monitoring SXR binding to DNA response elements in regulatory sequences that control expression of CYP2C8, CYP3A4 or MDR1 genes.

42. A method of claim 38 wherein said monitoring of SXR activity comprises monitoring SXR binding or SXR/RXR binding to nucleotide sequences that bind to SXR or to the SXR/RXR complex.

43. A method of claim 38 wherein said monitoring of SXR activity comprises monitoring SXR/RXR interaction.

44. A method of identifying drugs that do not modulate SXR activity which comprises screening drug candidates for their inability to:
(a) modulate the activity of SXR on expression levels of CYP2C8 or MDR1;
(b) modulate the expression of CYP3A4;
(c) modulate the expression of CYP2C8;
(d) modulate the expression of MDR1;
(e) modulate the expression of a synthetic reporter gene under the control of control elements responsive to SXR;
(f) modulate the expression of a chimeric gene, wherein the protein encoded by said chimeric gene maintains the ability to respond to SXR ligands;
(g) modulate SXR coactivator recruitment;
(h) modulate SXR corepressor displacement;
(i) modulate SXR binding to DNA response elements in regulatory sequences that control expression of CYP2C8, CYP3A4 or MDR1 genes; or
(j) modulate SXR/RXR interaction.

45. A drug identified by a method of any of claims 27-31, 34-37 or 39-44.

46. A method of screening patients to predict responsiveness to a pharmacologic agent, which comprises:
(a) obtaining a biological sample from said patient;
(b) screening said biological sample for an SXR parameter selected from the group consisting of SXR mRNA levels, SXR protein levels, SXR coactivator levels, SXR-coactivator interactions, SXR corepressor levels, SXR-corepressor interactions, SXR polymorphisms, SXR mutations, expression of an endogenous SXR regulated gene, and levels of an endogenous SXR ligand.

47. A method of claim 46 wherein said biological sample is screened for expression of an endogenous SXR regulated gene.

48. A method of claim 47 wherein said endogenous SXR regulated gene is a gene selected from the group consisting of CYP3A4 and CYP2C8.

49. A method of claim 46 wherein said responsiveness to a pharmacologic agent is responsiveness to a therapeutic effect.

50. A method of claim 46 wherein said responsiveness to a pharmacologic agent is responsiveness to a toxic effect.

51. A method of claim 44 wherein said responsiveness to a pharmacologic agent is responsiveness to a drug interaction.

52. A method of claim 44 wherein said pharmacologic agent is selected from the group consisting of an endogenous compound, a drug, an herbal compound and a nutrient.

53. A method of claim 44 wherein said biological sample is a tumor sample.

54. A method of claim 44 wherein said biological sample is a sample of normal cells or tissues, or a derivative thereof.

55. A method of drug chemotherapy which comprises coadministering a drug and an agent that modulates the activity or expression of SX.

56. A method of claim 53 which comprises coadministering a drug and an agent that downregulates the activity or expression of SXR.

57. A method of claim 53 which comprises coadministering a drug and an agent that upregulates the activity or expression of SXR.

58. A method of increasing the effectiveness of a drug which comprises coadministering said drug with an agent that modulates the actions of SXR.

59. A method of claim 53 wherein said agent is an SXR antagonist.

60. A method of claim 53 wherein said agent is an SXR agonist.

61. A method of claim 53 wherein said agent does not activate SXR.

62. A method of claim 53 wherein said drug is a taxane.
